(19) United States
(12) Patent Application Publication
(10) Pub. No.: US 2026/0053403 A1
(43) Pub. Date: Feb. 26, 2026

(54) BLOOD COLLECTION DEVICE AND METHOD OF MANUFACTURE

(71) Applicant: KPN INNOVATIONS LLC, LAKEWOOD, CO (US)

(72) Inventor: Kenneth Neumann, LAKEWOOD, CO (US)

(73) Assignee: KPN INNOVATIONS LLC, LAKEWOOD, CO (US)

(21) Appl. No.: 18/811,985

(22) Filed: Aug. 22, 2024

(51) Int. Cl. A61B 5/15

(52) U.S. Cl. CPC A61B 5/150977; A61B 5/150274

(57) ABSTRACT

A blood collection device including an array of needles configured to pierce an epidermal layer and extract a blood sample of a user, a collection channel positioned proximal to the array of needles and configured to transport the blood sample from the array of needles and a collection container configured to store the blood sample, wherein the collection container includes an inlet, wherein the inlet is configured to receive the blood sample and an outlet wherein the outlet is configured to expel the blood sample from the collection container.

### FIELD OF THE INVENTION

The present invention generally relates to the field of blood collection. In particular, the present invention is directed to a blood collection device and method of manufacture.

### BACKGROUND

Current blood collection devices utilize thick needles that penetrate in order to extract blood from a user's veins. As a result, current devices used for blood extraction can be painful and frightening to users. In addition, current blood collection devices that extract blood from capillaries may provide some relief, however they are limited in use.

### SUMMARY OF THE DISCLOSURE

In an aspect a blood collection device is described. The Blood collection device includes an array of needles configured to pierce an epidermal layer and extract a blood sample of a user, a collection channel positioned proximal to the array of needles and configured to transport the blood sample from the array of needles and a collection container configured to store the blood sample, wherein the collection container includes an inlet, wherein the inlet is configured to receive the blood sample and an outlet wherein the outlet is configured to expel the blood sample from the collection container.

In another aspect a method of manufacture of a blood collection device is described. Thie method includes the steps of receiving an array of needles configured to pierce an epidermal layer and extract a blood sample of a user, positioning a collection channel proximal to the array of needles, wherein the collection channel is configured to transport the blood sample from the array of needles and attaching a collection container to the collection channel wherein the collection container is configured to store the blood sample and wherein the collection container includes an inlet, wherein the inlet is configured to receive the blood sample and an outlet wherein the outlet is configured to expel the blood sample from the collection container.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIGS. 1A-B are illustrations of exemplary embodiments of a cross sectional view of a blood collection device;

FIG. 2A is an illustration of an exemplary embodiment of a front view of the blood collection device;

FIG. 2B is an illustration of an exemplary embodiment of a back view of the blood collection device;

FIGS. 3A-D are illustrations of exemplary embodiments of collection containers;

FIG. 4 is an illustration of an exemplary embodiment of a test card; and

FIG. 5 is a flow diagram illustrating an exemplary method of manufacture for the blood collection device.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods of manufacture for blood collection devices. In an aspect, embodiments of the present disclosure include an array of needles configured to puncture a user, a collection channel configured to receive a blood sample and a collection container configured to store the blood sample.

Aspects of the present disclosure can be used to collect blood samples without the use of a large needle. Aspects of the present disclosure can also be used to transfer blood samples onto test cards. Exemplary embodiments illustrating aspects of the present disclosure are described below in the context of several specific examples.

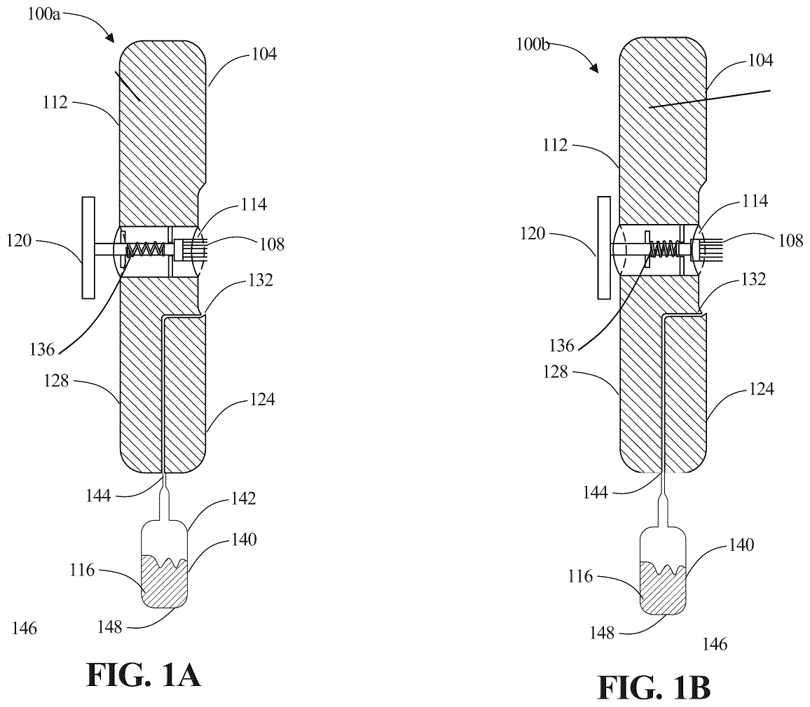

Referring now to FIGS. 1A-B, exemplary embodiments 100a-b of a cross sectional view of a blood collection device 104 is illustrated. In one or more embodiments, blood collection device 104 may include a needle. A “Needle” for the purposes of this disclosure is a slender pointed device used for puncturing tissue. For example, and without limitation, needle may include a slender stainless steel device configured to puncture the skin of a user and cause blood to be expelled. In one or more embodiments, needle may include a slender pointed device user for penetration of one or more objects. In one or more embodiments, needle may include a metallic object configured to piece a kin of an individual. In one more embodiments, needle may include a material such as steel, stainless steel, aluminum, gold, silver, coppers and/or the like. In one or more embodiments, needle may include a slim metallic device with a pointed end configured to pierce an epidermal layer of user. An “epidermal layer” as described in this disclosure refers to the outermost layer of the skin. In one or more embodiments, the epidermal layer may serve as a protective barrier against environmental hazards such as pathogens, chemicals and the like. In one or more embodiments, the epidermal layer may surround a dermal layer. In one or more embodiments, capillary blood may be located in the dermal Layer. In one or more embodiments, needle may be configured to pierce a dermal layer as well. In one or more embodiments, needle may include a length of 50 to 900 micrometers. In one or more embodiments, needle may contain a length of 50 to 3000 micrometers. In one or more embodiments, needle may include a length of between 1 and 2.25 millimeters. In one or more embodiments, needle may include a diameter of 1 to 25 micrometers. In one or more embodiments, needle may include a gauge of between 21 and 30. In one or more embodiments, needles may be substantially cylindrical wherein a predominant portion of needle may be cylindrical in shape. In one or more embodiments, needle may include a hollow center, wherein a liquid may pass through a center of needle. In one or more embodiments, needle may include and/or be included in a lancet. A “lancet” for the purposes of this disclosure is an incision device configured to puncture the skin in order to obtain capillary blood from one or more capillaries. In one or more embodiments, lancet may include a spring loaded mechanism configured to puncture skin of a user. In one or more embodiments, lancet may include a needle configured to puncture skin of a user. In one or more embodiments, spring loaded mechanism may drive needle quickly into skin of user.

With continued reference to FIGS. 1A-B, blood collection device 104 may include an array of needles 108. An “array of needles” for the purposes of this disclosure refers to more than one needles positioned proximal to one another. For example, and without limitation, array of needles 108 may include plurality of needles positioned in the same direction, a plurality of needles positioned in a square grid, a plurality of needles positioned in a circular formation and the like. In one or more embodiments, array of needles 108 may be configured to pierce at least an epidermal layer of a user in order to extract a blood sample 116 of user. A “user” for the purposes of this disclosure is an individual using, or interacting with, blood collection device 104. For example, and without limitation, user may include a diabetic individual attempting to extract blood in order to measure the blood sugar levels. In one or more embodiments, user may include an individual attempting to extract their own blood. In one or more embodiments, user may include an individual attempting to extract blood form a patient, such as for example, a medical professional, a friend of the patient, a family member of the patient and the like. In one or more embodiments, array of needles 108 may be configured to pierce epidermal layer and reach capillary beds of user. A “capillary bed” for the purposes of this disclosure is a network of small blood vessels in the body that connect small arteries to small veins. In one or more embodiments, capillaries may transport oxygen, nutrients and blood cells into the body's systems or organs. In one or more embodiments, array of needles 108 may puncture a skin of an individual in order to access capillaries beneath the skin. In one or more embodiments, array of needles 108 may be configured to puncture capillaries of a user, such that blood is released through skin of the user. In one or more embodiments, array of needles 108 may be configured to puncture at least epidermal layer in order to extract blood sample 116. A “blood sample” for the purposes of this disclosure refers to blood that has been extracted from user for the purposes of testing. For example, and without limitation, blood sample 116 may include blood that has been extracted to determine an allergy, to determine sugar levels, to determine nutrient levels and the like. In one or more embodiments, array of needles 108 may be configured to extract blood sample 116 from user. In one or more embodiments, array of needles 108 may puncture user's skin wherein blood within capillaries may be released. In one or more embodiments puncturing user's skin may include puncturing one or more capillaries beneath the user's skin.

With continued reference to FIGS. 1A-B, array of needles 108 may be encased within a housing 112 of blood collection device 104. In one or more embodiments, blood collection device 104 may include a housing 112 configured to encapsulate components of blood collection device 104, such as array of needles 108. In one or more embodiments, in a retracted position, array of needles 108 may be located within a perimeter of housing 112. In one or more embodiments, housing 112 may include a cavity 114 in which array of needles 108 may move into and out of perimeter of housing 112. In one or more embodiments, array of needles 108 may be located within a perimeter of housing 112. In one or more embodiments, when moved, array of needles 108 may move outside a perimeter of housing 112 and/or through cavity 114. In one or more embodiments, when moved, array of needles 108 may extend past a surface of housing 112. In one or more embodiments, in an extended position, array of needles 108 may extend past a lower surface 124 of housing 112 in order to puncture skin of user.

With Continued reference to FIGS. 1A-B, blood collection device 104 may be configured to receive capillary blood. In one or more embodiments, blood collection device 104 may be configured to receive venous blood. “Venous blood” for the purposes of this disclosure is deoxygenated blood that flows from tissues into the heart through the veins. In one or more embodiments, array of needles 108 may include one or more needle configured to puncture the veins of the user in order to capture venous blood. In one or more embodiments, the one or more needles may include a hypodermic needle configured to extract blood from one or more veins of a user. In one or more embodiments, the hypodermic needle may puncture the vein of a user wherein blood may flow through the hypodermic needle and into blood collection device. In one or more embodiments, a collection channel (as described in further detail blow) may be configured to receive the blood form the hypodermic needle.

With continued reference to FIG. 1, in one or more embodiments, housing may include a material such as plastic, ABS plastic, hard plastic, glass, metal, aluminum and the like. In one or more embodiments, housing 112 may be manufactured through a molding process wherein housing 112 may be molded. In one or more embodiments, housing may contain two parts wherein each part may be manufactured through a molding process. In one or more embodiments, components of blood collection device may be placed within one part of housing and encased within housing by combining the two parts together. In one or more embodiments, a molding process may include an injection molding process. In one or more embodiments housing 112 may include two parts wherein a first part and a second part are both created in a molding process. In one or more embodiments, housing 112 may be assembled by combining first part and second part. In one or more embodiments, following a molding process, first part and second part may be secured together using an adhesive

With continued reference to FIGS. 1A-B, in one or more embodiments, blood collection device 104 may include a push lever 120 mechanically connected to array of needles 108. In one or more embodiments, push lever 120 may include a switch, a push button, a pull down lever and the like configured to drive array of needles 108 into skin of user. In one or more embodiments, push lever 120 may be rigidly attached to array of needles 108 wherein a pressure of push lever 120 may result in movement of array of needles 108. In one or more embodiments, push lever 120 may be rigidly attached to array of needles 108 wherein movement of push lever 120 over a particular distance may result in movement of array of needles 108 over a substantially similar distance. In one or more embodiments, push lever 120 may cause movement of array of needles 108 into and/or out of housing 112. In one or more embodiments, a pressing of push lever 120 may cause array of needles 108 to move outside of housing 112. In one or more embodiments, push lever 120 may include a spring 136 configured to provide a force to return array of needles 108 to an original position. In one or more embodiments, in an original position, array of needles 108 may be located within a perimeter of housing 112. In one or more embodiments, actuation and/or pressing of push lever 120 may cause array of needles 108 to extend past a perimeter of housing 112 and/or past a lower surface 124 of housing 112. In one or more embodiments, removal of a force applied to push lever 120 (e.g. the user stops pressing) may result in array of needles 108 retracting back into a perimeter of housing 112 due to a force applied by spring 136. In one or more embodiments, in an original position array of needles 108 may be located within a perimeter of housing 112. In one or more embodiments, when a force is applied into push lever 120, array of needles 108 extends past lower surface 124 and/or perimeter of housing 112. In one or more embodiments, removal of a force on push lever 120 may result in retract of array of needles 108 back into perimeter of housing 112.

With continued reference to FIGS. 1A-B, push lever 120 may be located at or near an upper surface 128 of housing 112. In one or more embodiments, a push button, lever and/or switch of lever may be located at or near upper surface 128 of housing 112. In one or more embodiments, push lever 120 may contain an arm extending into housing 112. In one or more embodiments, the arm may be rigidly attached to array of needles 108. In one or more embodiments, a pressing of push lever 120 may extend arm into housing 112, and as a result, array of needles 108 may extend outside of housing 112 and/or past a lower surface 124 of housing 112. In one or more embodiments, movement of arm may cause compression of spring 136, wherein release of push lever 120 may cause expansion of spring 136 and a resulting movement of arm in an opposing direction. In one or more embodiments, when pressed, push lever 120 may be configured to period epidermal layer of user using array of needles 108.

With continued reference to FIG. 1, array of needles 108 may be controlled using an actuator. An actuator may include a component of a machine that is responsible for moving and/or controlling a mechanism or system. An actuator may, in some cases, require a control signal and/or a source of energy or power. In some cases, a control signal may be relatively low energy. Exemplary control signal forms include electric potential or current, pneumatic pressure or flow, or hydraulic fluid pressure or flow, mechanical force/torque or velocity, or even human power. In some cases, an actuator may have an energy or power source other than control signal. This may include a main energy source, which may include for example electric power, hydraulic power, pneumatic power, mechanical power, and the like. In some cases, upon receiving a control signal, an actuator responds by converting source power into mechanical motion. In some cases, an actuator may be understood as a form of automation or automatic control.

With continued reference to FIG. 1, in some embodiments, actuator may include a hydraulic actuator. A hydraulic actuator may consist of a cylinder or fluid motor that uses hydraulic power to facilitate mechanical operation. Output of hydraulic actuator may include mechanical motion, such as without limitation linear, rotatory, or oscillatory motion. In some cases, hydraulic actuator may employ a liquid hydraulic fluid. As liquids, in some cases, are incompressible, a hydraulic actuator can exert large forces. Additionally, as force is equal to pressure multiplied by area, hydraulic actuators may act as force transformers with changes in area (e.g., cross sectional area of cylinder and/or piston). An exemplary hydraulic cylinder may consist of a hollow cylindrical tube within which a piston can slide. In some cases, a hydraulic cylinder may be considered single acting. Single acting may be used when fluid pressure is applied substantially to just one side of a piston. Consequently, a single acting piston can move in only one direction. In some cases, a spring may be used to give a single acting piston a return stroke. In some cases, a hydraulic cylinder may be double acting. Double acting may be used when pressure is applied substantially on each side of a piston; any difference in resultant force between the two sides of the piston causes the piston to move.

With continued reference to FIG. 1, in some embodiments, actuator may include a pneumatic actuator. In some cases, a pneumatic actuator may enable considerable forces to be produced from relatively small changes in gas pressure. In some cases, a pneumatic actuator may respond more quickly than other types of actuators, for example hydraulic actuators. A pneumatic actuator may use compressible fluid (e.g., air). In some cases, a pneumatic actuator may operate on compressed air. Operation of hydraulic and/or pneumatic actuators may include control of one or more valves, circuits, fluid pumps, and/or fluid manifolds.

With continued reference to FIG. 1, in some cases, actuator may include an electric actuator. Electric actuator may include any electromechanical actuators, linear motors, and the like. In some cases, actuator may include an electromechanical actuator. An electromechanical actuator may convert a rotational force of an electric rotary motor into a linear movement to generate a linear movement through a mechanism. Exemplary mechanisms, include rotational to translational motion transformers, such as without limitation a belt, a screw, a crank, a cam, a linkage, a scotch yoke, and the like. In some cases, control of an electromechanical actuator may include control of electric motor, for instance a control signal may control one or more electric motor parameters to control electromechanical actuator. Exemplary non-limitation electric motor parameters include rotational position, input torque, velocity, current, and potential. electric actuator may include a linear motor. Linear motors may differ from electromechanical actuators, as power from linear motors is output directly as translational motion, rather than output as rotational motion and converted to translational motion. In some cases, a linear motor may cause lower friction losses than other devices. Linear motors may be further specified into at least 3 different categories, including flat linear motor, U-channel linear motors and tubular linear motors. Linear motors may be directly controlled by a control signal for controlling one or more linear motor parameters. Exemplary linear motor parameters include without limitation position, force, velocity, potential, and current.

With continued reference to FIG. 1, in some embodiments, an actuator may include a mechanical actuator. In some cases, a mechanical actuator may function to execute movement by converting one kind of motion, such as rotary motion, into another kind, such as linear motion. An exemplary mechanical actuator includes a rack and pinion. In some cases, a mechanical power source, such as a power take off may serve as power source for a mechanical actuator. Mechanical actuators may employ any number of mechanism, including for example without limitation gears, rails, pulleys, cables, linkages, and the like.

With continued reference to FIG. 1, actuator may be configured to translate array of needles 108 into a user. In one or more embodiments, push lever 120 may instantiate actuator wherein a pressing of push lever may signify to actuator to cause array of needles 108 to be directed into user. In one or more embodiments, actuator may allow for quick insertion and removal of array of needles 108 such that pain may be minimal to user. In one or more embodiments, actuator may press array of needles 108 into user in instances in which a user may be afraid to manually prick themselves. In one or more embodiments, actuator may allow for a means in which a user is pricked by array of needles with the user manually inserting array of needles 108 into their skin. In one or more embodiments, actuator may control movement of array of needles wherein movement of actuator may result in movement of array of needles 108.

With continued reference to FIGS. 1A-B, blood collection device 104 contains a collection channel 132 positioned proximal to array of needles 108. A “collection channel,” for the purposes of this disclosure, refers to a conduit that is configured to collect blood sample 116. In one or more embodiments, collection channel 132 may be configured to receive blood sample 116 from user. In one or more embodiments, collection channel 132 may be configured to collect blood sample 116 extracted from user following penetration of array of channels into user. In one or more embodiments, collection channel 132 may include an opening configured to receive blood sample 116. In one or more embodiments, the opening may range from between 5-10 millimeters. In one or more embodiments, the opening may range from 5-50 millimeters. In one or more embodiments, the opening may range from between 5-100 millimeters. In one or more embodiments, collection channel 132 may be positioned proximal to array of needles 108 wherein blood sample 116 extracted by array of needles 108 may be collected by collection channel 132. In one or more embodiments, collection channel 132 may be located on lower surface 124 of housing 112. In one or more embodiments, collection channel 132 may include a plurality of channels. In one or more embodiments, collection channel 132 may be etched and/or molded into blood collection device 104 and/or housing 112. In one or more embodiments, blood collection device 104 may include housing 112 in order to encapsulate one or more components of blood collection device 104. In one or more embodiments, collection channels 132 may be etched and/or molded into housing 112 of blood collection device 104. In one or more embodiments, collection channel 132 may include a circular channel surrounding array of needles 108 wherein blood sample 116 extracted by array of needles 108 may be collected by circular channel. In one or more embodiments, collection channel 132 may guide blood sample 116 from collection site to a collection area. In one or more embodiments, collection channel 132 may include pathways for blood sample 116 to travel from a puncture site to a collection area. In one or more embodiments, puncture sit may include a location on user in which array of needles 108 has punctured user's skin. In one or more embodiments, collection channel 132 contain openings at and/or near puncture site in order to collect blood sample 116 following puncture of user's skin. In one or more embodiments, collection channel 132 may be made of silicon, plastic and/or a combination of materials. In one or more embodiments, collection channel 132 may be situated at or near an area in which blood collection device 104 comes into contact with user. In one or more embodiments, collection channel 132 may navigate blood sample 116 from puncture site to a collection area. In one or more embodiments, blood sample 116 may travel to cavity 114 within blood collection device 104 wherein collection channel 132 may be fluidly connected to the cavity 114 and transport blood sample 116 from circular cavity 114 to a collection area. In one or more embodiments, collection channel 132 may be positioned proximally to array of needles 108 and configured to transport the blood sample 116 from the array of needles 108. In one or more embodiments, array of needles 108 may puncture capillaries of user wherein released blood may be collected by collection channel 132. In one or more embodiments, lower surface 124 of housing 112 may be placed substantially in contact with user, wherein a pressing of push lever 120 may cause array of needles 108 to extend past lower surface 124 and into user.

With continued reference to FIGS. 1A-B, blood collection device 104 may include a strap, suction cups and the like in order to secure blood collection device 104 and/or a portion thereof to user. In one or more embodiments, blood collection device 104 may be strapped and/or attached a portion of user's body wherein a pressing of push lever 120 may result in puncturing of array of needles 108 into skin of user. In one or more embodiments, lower surface 124 of housing 112 may include an adhesive configured to temporarily adhere to a skin of user. In one or more embodiments, lower surface 124 of housing 112 may include suction cups configured to provide a suction force and/or vacuum on user's body. In one or more embodiments, adhesives, straps and the like may ensure a tight seal between user and blood collection device 104. In one or more embodiments, adhesive, straps and the like may ensure that blood sample 116 is collected by blood collection device 104 and does not seep through and down user's body. In one or more embodiments, strap, suction cups, adhesive and the like may provide an airtight seal between housing 112 and a user's skin. In one or more embodiments, airtight seal may ensure that blood sample 116 does not leak from blood collection device 104.

With continued reference to FIGS. 1A-B, collection channel 132 may collect blood sample 116 and transport blood sample 116 to a collection container 140. A “collection container,” for the purposes of this disclosure, is a device configured to store a fluid. For example, and without limitation, collection container 140 may store blood sample 116. In one or more embodiments, collection container 140 may contain a nonporous material configured to store a fluid without leaking. In one or more embodiments, collection container 140 may be configured to receive and/or store blood sample 116. In one or more embodiments, collection container 140 may be located outside of housing 112. In one or more embodiments, collection container 140 may be removably attached to housing 112. “Removably attached,” for the purposes of this disclosure, refers to a configuration in which two components may be attached and detached multiples times without damage occurring to either of two components. For example, and without limitation, two components may be removably attached wherein one component may be unscrewed and re-screwed from a second component. In one or more embodiments, collection container 140 may be removably attached to housing 112 wherein collection container 140 may be separated and re-attached to housing 112. In one or more embodiments, collection container 140 may be removably attached wherein collection container 140 may be secured to housing 112 and/or blood collection device 104 through a screwing and unscrewing process. In one or more embodiments, collection container 140 may contain indents or grooves that are configured to allow collection container 140 to be screwed and secured to housing 112 and/or blood collection device 104. In one or more embodiments, collection container 140 and/or housing 112 may contain threads which allow for securement of collection container 140 to housing 112. In one or more embodiments, collection container 140 may be removably attached through the use of magnetic components. In one or more embodiments, collection container 140 and housing 112 may both contain magnetic components which allow for temporary securement to housing 112. In one or more embodiments, collection container 140 may be removably attached to blood collection device 104 and/or housing 112 wherein collection container 140 may be removed from housing 112 following use of blood collection device 104.

With continued reference to FIGS. 1A-B, in one or more embodiments, collection container 140 may include a container in the shape of a vile, a container in the shape of a cup, a container having a U-shape, a cylindrical container and the like. In one or more embodiments, collection container 140 may include any shape in which collection container 140 may hold a fluid such as blood sample 116.

With continued reference to FIGS. 1A-B, collection container 140 may contain a first end 144 and a second end 148. In one or more embodiments, collection container 140 may be removably attached to housing 112 at first end 144. In one or more embodiments, first end 144 may include an inlet. In on or more embodiments, inlet may include an opening configured to receive blood sample 116. In one or more embodiments, threads, grooves and/or any other securing mechanisms may be located at or near first end 144 wherein the securing mechanisms may be configured to secure first end 144 to housing 112. In one or more embodiments, blood sample 116 may travel from collection channel 132, through inlet and into collection container 140. In one or more embodiments, during extraction of blood sample 116, collection channel 132 may be located above collection container 140, wherein blood sample 116 may flow through collection channel 132 and into collection container 140 using gravity.

With continued reference to FIGS. 1A-B, collection container 140 may include inlet and outlet. In one or more embodiments, inlet may include an opening configured to receive blood sample 116 and outlet may include an opening configured to expel blood sample 116. In one or more embodiments, inlet may be configured to receive blood sample 116 during extraction of blood sample 116. In one or more embodiments, inlet may be configured to receive blood sample 116, wherein collection container 140 may be configured to store blood sample 116 during at least an extraction process. In one or more embodiments, collection container 140 may include outlet. In one or more embodiments, blood sample 116 may be released and/or expelled from outlet. In one or more embodiments, outlet and inlet may be situated and/or located at first end 144. In one or more embodiments, outlet and inlet may contain the same opening. In one or more embodiments, outlet may contain a differing opening on collection container 140 as inlet. In one or more embodiments, outlet may be located on an opposing end of inlet. In one or more embodiments inlet is configured to receive the blood sample 116 and outlet is configured to expel blood sample 116 from the collection container 140.

With continued reference to FIGS. 1A-B, collection container 140 may further include second end 148. In one or more embodiments, second end 148 may be located on an opposing end of first end 144 on collection container 140. In one or more embodiments, second end 148 may include a closed end wherein blood sample 116 may not travel through second end 148. In one or more embodiments, second end 148 may include an opening in which blood sample 116 may be expelled and/or released through second end 148. In one or more embodiments, outlet may be situated at or near second end 148. In one or more embodiments, outlet may be situated at or near second end 148 wherein blood sample 116 may be expelled and/or released through second end 148. In one or more embodiments, second end 148 may be situated at the most distal portion of collection container 140 in comparison to housing 112. In one or more embodiments, first end 144 may be smaller in diameter than second end 148. In one or more embodiments, a diameter and/or perimeter of collection container 140 may increase from first end 144 in a direction of second end 148. In one or more embodiments, collection container 140 may be predominantly cylindrical wherein first end 144 of collection container 140 may be smaller in diameter than second end 148.

With continued reference to FIGS. 1A-B, in one or more embodiments collection container 140 may include a pliable material. A “pliable material” for the purposes of this disclosure is a material that can bend, fold, twist, or be manipulated without causing permanent deformations to the material. For example, and without limitation, pliable material may include silicon wherein a force applied onto collection container 140 may not cause permanent deformations to collection container 140. In one or more embodiments, pliable material may include but is not limited to, silicone rubber, low density polyethylene, a soft plastic, rubber, polycarbonate, silicone, vinyl and the like. In one or more embodiments, collection container 140 may include a pliable material wherein collection container 140 may be squeezed in order to expel blood sample 116 from first container. In one or more embodiments, a portion of collection container 140 may include pliable material, wherein the portion of collection container 140 having pliable material may be pressed on and/or squeezed in order to expel blood sample 116 out of collection container 140. In one or more embodiments, a portion of collection container 140 situated at or near second end 148 may include pliable material, wherein collection container 140 may be pressed or squeezed at second end 148 in order to expel blood sample 116 out of first end 144. In one or more embodiments, a portion of collection container 140 at or near first end 144 may include a nonpliable material, such as but not limited to, glass, hard plastic and the like.

With continued reference to FIGS. 1A-B, in one or more embodiments, collection container 140 may include a Pasteur pipette 142. In one or more embodiments, collection container 140 may predominantly take the shape of Pasteur pipette 142. A “Pasteur pipette” also known as a “dropper” or a transfer pipette“ for the purposes of this disclosure is a device used to transfer small quantities of liquid. In one or more embodiments, Pasteur pipette 142 may include a stem connected to a bulb, wherein a fluid may be stored in the bulb and transferred through the stem. In one or more embodiments, the bulb may include a pliable material such that a pressing or squeezing of the stem may control of the flow of a fluid through the stem. In one or more embodiments, Pasteur pipette 142 may be configured to transfer small quantities of blood sample 116 from collection container 140 to another device for laboratory testing. In one or more embodiments, a stem of Pasteur pipette 142 may be located at or near first end 144 whereas a bulb of Pasteur pipette 142 may be located at or near second end 148 of collection container 140. In one or more embodiments, inlet may be located on and/or near stem. In one or more embodiments, outlet may also be located at or near stem. In one or more embodiments, a bulb of Pasteur pipette 142 may include a pliable material wherein blood sample 116 may be expelled through stem by pressing on bulb. In one or more embodiments, a portion of stem of Pasteur pipette 142 may be inserted into housing 112 and configured to receive blood sample 116. In one or more embodiments, a stem of Pasteur pipette 142 may include threading configured to secure stem to housing 112.

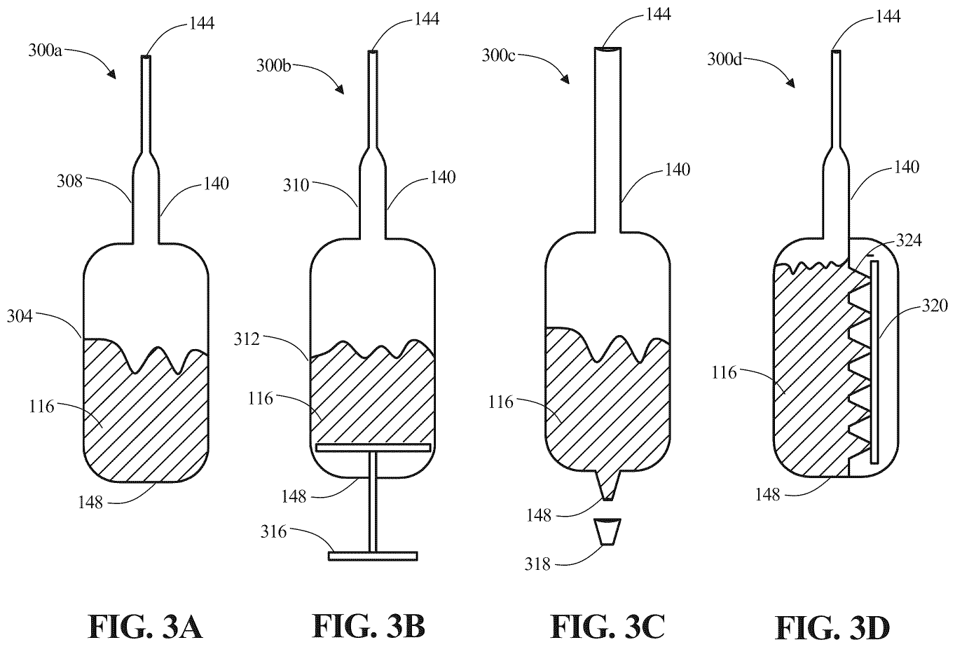

Referring to FIG. 3C, collection container 140 may include a removable cap 318 located at outlet. In one or more embodiments, removable cap 318 may include a component configured to prevent fluid from leaving through outlet. In one or more embodiments, second end 148 may include threading wherein removable cap 318 may be twisted and/or secured onto second end 148. In one or more embodiments, blood sample 116 may be expelled through outlet when removable cap 318 is removed. In one or more embodiments, removable cap 318 may prevent unwanted expelling of blood sample 116. In one or more embodiments, removable cap 318 may temporarily close second end 148 until user desires to expel blood sample 116 from second end 148. In one or more embodiments, removable cap 318 may allow for removable and securement of removable cap onto collection container 140. In one or more embodiments, removable cap 318 may be configured to prevent release of the blood sample 116 from the outlet. In one or more embodiments, removable cap may include a screw cap, a snap fit, a push on, a pressure sealed cap, a magnetic cap, a screw and bayonet cap, a clamped cap, a twist off, and the like. In one or more embodiments, removable cap may contain a magnetic element that is configured to magnetically attract to a magnetic element located at second end 148. In one or more embodiments, removable cap 318 may be magnetically attached to collection container 140 through the use of magnetic elements. In one or more embodiments, removable cap 318 may contain threading that is configured to twist onto threading located at second 148. This may be explained in further detail below.

Referring now specifically to FIG. 1A, an exemplary embodiment 100a of blood collection device 104 in an unpressed position is described. In one or more embodiments, blood collection device 104 may be an unpressed position wherein array of needles 108 is located within a perimeter of housing 112. In one or more embodiments, in unpressed position, spring 136 may be in an uncompressed state wherein there is no potential energy stored within spring 136.

Referring now specifically to FIG. 1B, an exemplary embodiment 100b of blood collection device 104 in a pressed position is described. In one or more embodiments, in a pressed position array of needles 108 may extend past lower surface 124 of housing 112. In one or more embodiments, in a pressed position, array of needles 108 may extend outside a perimeter of housing 112. In one or more embodiments, a pressing of push lever 120 may result in compression of string and extension of array of needles 108. In one or more embodiments, release of push lever 120 may result in uncompress ion of spring 136 and retraction of array of needles 108. In one or more embodiments, collection channel 132 may be situated proximal to array of needles 108 wherein blood expelled by array of needles 108 may be collected and transmitted through collection channel 132. In one or more embodiments, blood sample 116 may travel through collection channel 132 and into collection container 140.

With continued reference to FIG. 1, collection channel 132 may be powered using a pump. Pump may include a substantially constant pressure pump (e.g., centrifugal pump) or a substantially constant flow pump (e.g., positive displacement pump, gear pump, and the like). Pump can be hydrostatic or hydrodynamic. As used in this disclosure, a “pump” is a mechanical source of power that converts mechanical power into fluidic energy. A pump may generate flow with enough power to overcome pressure induced by a load at a pump outlet. A pump may generate a vacuum at a pump inlet, thereby forcing fluid from a reservoir into the pump inlet to the pump and by mechanical action delivering this fluid to a pump outlet. Hydrostatic pumps are positive displacement pumps. Hydrodynamic pumps can be fixed displacement pumps, in which displacement may not be adjusted, or variable displacement pumps, in which the displacement may be adjusted. Exemplary non-limiting pumps include gear pumps, rotary vane pumps, screw pumps, bent axis pumps, inline axial piston pumps, radial piston pumps, and the like. Pump may be powered by any rotational mechanical work source, for example without limitation and electric motor or a power take off from an engine. Pump may be in fluidic communication with at least a reservoir and/or collection channel 132. In some cases, reservoir and/or collection channel 132 may be unpressurized and/or vented. Alternatively, reservoir may be pressurized and/or sealed. In one or more embodiments, pump may allow for blood sample 116 to be transported from collection channel 132 to collection container 140. In one or more embodiments, pump may create a vacuum within cavity 110. In one or more embodiments, in instances in which housing 112 is placed against an individual's arm, cavity may be isolated from surrounding air. In one or more embodiments, pump may be configured to create negative pressure within cavity 110 such that the negative pressure causes blood sample 116 to travel through collection channel 132. In one or more embodiments, pump may be located within housing and powered by an electric motor. In one or more embodiments, pump may be configured to create a pressure differential between collection container and collection container and/or cavity 114 wherein blood sample 116 may travel from cavity 110 or collection channel and into collection container. In one or more embodiments, pump may cause a pressure drop between cavity 110 within housing 112 and a user wherein housing may be attached to user through suction. In one or more embodiments, a negative pressure differential within cavity may cause suction of capillary blood from user. For example, and without limitation, a user may be pricked in the arm by array of needles 108 wherein the negative pressure differential between the user's arm and housing may cause capillary blood to be sucked out of puncture holes on the user's arm. In one or more embodiments, collection channel may contain a valve to ensure that blood sample does not flow from collection container into cavity. As used in this disclosure, a “valve” is a component that controls fluidic communication between two or more components. Exemplary non-limiting valves include directional valves, control valves, selector valves, multi-port valves, check valves, and the like. Valves may include any suitable valve construction including ball valves, butterfly valves, needle valves, globe valves, gate valves, wafer valves, regulator valves, and the like. Valves may be included in a manifold of hydraulic or pneumatic circuit, for example allowing for multiple ports and flow paths. Valves may be actuated by any known method, such as without limitation by way of hydraulic, pneumatic, mechanical, or electrical energy. For instance, in some cases, a valve may be actuated by an energized solenoid or electric motor. Valve actuators and thereby valves themselves, may be controlled by a computing device. A computing device may be in communication with valve, for example by way of one or more of electrical communication, hydraulic communication, pneumatic communication, mechanical communication, and the like.

With Continued reference to FIG. 1, collection container 116 may be oriented below collection channel 132 wherein gravity may cause blood sample 116 to flow downwards into collection container 132. In one or more embodiments, a puncture site created by array of needles may be located above collection container 140 wherein collection channel 132 may collect blood sample and blood sample may flow downwards into collection container 140. In one or more embodiments, collection channel 132 may include a capillary tube. A “capillary tube” for the purposes of this disclosure is a narrow hollow tube that utilizes capillary action in order to draw and transport small volumes of liquid. In one or more embodiments, capillary tube may contain an internal diameter of less than 1 millimeter (mm). In one or more embodiments, capillary tube may include a material such as glass or plastic. In one or more embodiments, the narrow diameter of capillary tube may allow for blood sample to be collected using capillary action. In one or more embodiments, blood sample 116 may flow through collection channel 132 through capillary action. In one or more embodiments, capillary tube may be coated with an anticoagulant such as heparin in order to prevent blood sample 116 from collecting during transportation to collection container 140. In one or more embodiments, capillary action within capillary tube may allow for blood sample to be collected and rise within collection channel without the need of external force. In one or more embodiments, a user may be instructed to orient blood collection device 100 in a direction such that collection container may be oriented below housing. In one or more embodiments, blood sample may then travel downward through collection channel 132 and into collection container. In one or more embodiments, blood collection device 100 may include pumps as described above wherein a pump may apply a force or pressure on blood sample such that the force or pressure causes blood sample to flow through collection channel 132 and into collection container 140.

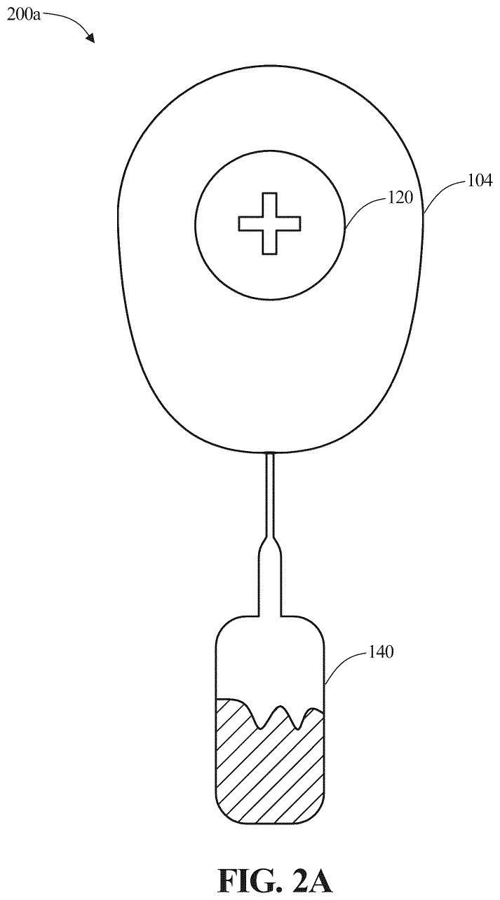

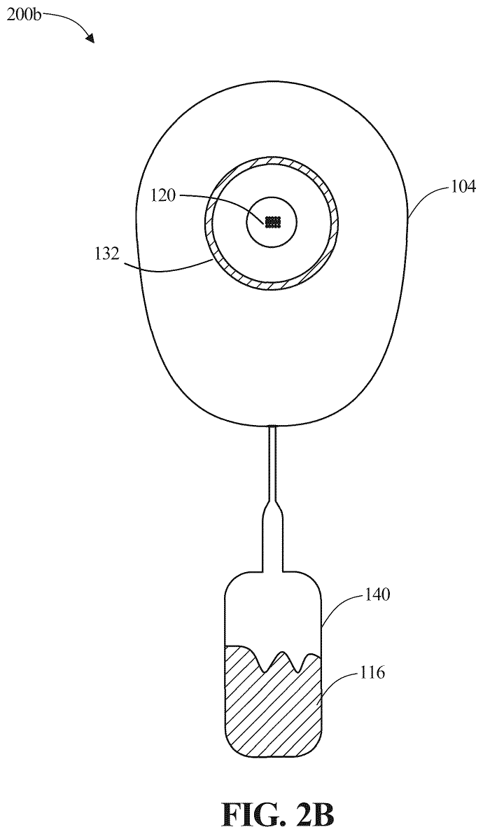

Referring now to FIG. 2A, an exemplary embodiment 200a of a front view of blood collection device 104 is described. In one or more embodiments, blood collection device 104 may include a push lever 120. A “push lever” for the purposes of this disclosure is a component that translates a force applied onto it in linear motion. For example, and without limitation, a user may apply a force onto push lever wherein the force applied onto push lever may cause array of needles to extend linearly a predetermined distance. In one or more embodiments, push lever may include a push button wherein the pressing of push button may cause array of needles 108 to extend into a skin of user. In one or more embodiments, upon pressing of push lever 120, array of needles 108 may retract, and blood sample 116 may be collection by collection channel 132 and stored into collection container 140.

Referring now to FIG. 2B, an exemplary embodiment 200b of a back view of blood collection device 104 is described. In one or more embodiments, array of needles 108 may be situated near lower surface 124 of blood collection device 104. In one or more embodiments, array of needles 108 may extend past lower surface 124 when push lever 120 is pressed or engaged. In one or more embodiments, collection channel 132 may be located around array of needles 108 wherein collection channel 132 may be configured to collection blood sample 116 expelled as a result of array of needles 108. In one or more embodiments collection channel 132 may include a circular channel configured to accept blood sample 116. In one or more embodiments, collection channel 132 may transmit blood sample 116 from lower surface 124 to inside housing 112 and into collection container 140. In one or more embodiments, collection channel 132 may be situated at below array of needles 108 wherein blood sample 116 may travel downward towards collection channel 132 due to gravity.

Referring now to FIG. 3A, an exemplary embodiment 300a of a collection container 140 is described. In one or more embodiments, collection container 140 may contain a bulb 304 and a stem 308 similar to that of a Pasteur pipette. In one or more embodiments, first end 144 of collection container 140 may include a stem, while bulb may be situated at or near second end 148. In one or more embodiments, collection container 140 may be rotated to allow blood sample 116 to drip out onto a surface such as a test card (as described in further detail below). In one or more embodiments, collection container 140 may include a pliable material, wherein the pliable material may allow for temporary deformation of collection container 140. In one or more embodiments, pliable material may allow for collection container 140 to be squeezed, wherein blood sample 116 may be expressed out of collection container 140.

Referring now to FIG. 3B, another exemplary embodiment 300b of collection container 140 is described. In one or more embodiments, collection container 140 may include a syringe 310. A “syringe 310” as used in this disclosure is a device use to inject fluids. In one or more embodiments, syringe 310 may include a barrel 312 configured to store a fluid such as blood sample 116. In one or more embodiments, barrel 312 may include an outlet in which fluids may be expelled through. In one or more embodiments, syringe 310 may further include a plunger 316 configured to push a fluid through outlet of barrel 312. In one or more embodiments, plunger 316 may include a plunger 316 flange wherein the plunger 316 flange is configured to be gripped by user. In one or more embodiments, plunger 316 may be configured to control a flow of fluid such as blood sample 116 out of barrel 312. In one or more embodiments, plunger 316 may include a seal in order to prevent a fluid from leaking out of a wrong side of barrel 312. In one or more embodiments, barrel 312 may include first end 144 and second end 148 wherein first end 144 may include an inlet and outlet and second end 148 may include an opening in which plunger 316 and/or a portion thereof may pass through. In one or more embodiments, seal may prevent a fluid such ass blood sample 116 from leaking through second end 148. In one or more embodiments, blood sample 116 may be received by housing 112 and/or blood collection device 104 through first end 144 and expel blood sample 116 through outlet situated at the same end or opening as inlet. In one or more embodiments, pressing plunger 316 in a direction towards first end 144 may cause blood sample 116 to move in a direction towards first end 144 and out of barrel 312. In one or more embodiments, syringe 310 may allow for control of a flow of blood sample 116. In one or more embodiments, blood sample 116 may be placed on a test card wherein syringe 310 may control the flow of blood sample 116 onto test card. In one or more embodiments, collection container 140 includes a syringe 310, wherein the syringe 310 is configured to receive the blood sample 116 at the inlet and expel the blood sample 116 at the outlet

Referring now to FIG. 3C, yet another exemplary embodiment 300c of collection container 140 is described. In one or more embodiments, collection container 140 may contain first end 144 and second end 148. In one or more embodiments, first end 144 may include inlet wherein blood sample 116 may flow from blood collection device 104 and/or housing 112 into inlet. In one or more embodiments, collection container 140 may further include outlet situated at or near second end 148 wherein blood sample 116 may exit collection container 140 through outlet. In one or more embodiments, outlet may include an opening situated at or near second end 148. In one or more embodiments, removable cap 318 may be situated at or near second end 148 wherein removable cap 318 may control a flow of blood sample 116 through second end 148 and/or outlet. In one or more embodiments, removable cap 318 may contain threads wherein removable cap 318 may be twistedly tightened at or near second end 148. In one or more embodiments, during blood collection, removable cap 318 may prevent blood sample 116 from exiting collection container 140. In one or more embodiments, following collection of blood sample 116, collection container 140 may be removed from housing 112, wherein blood sample 116 may be removed from collection container 140 through second end 148. In one or more embodiments, removable cap 318 may be untightened and removed such that blood sample 116 may exit through second end 148.

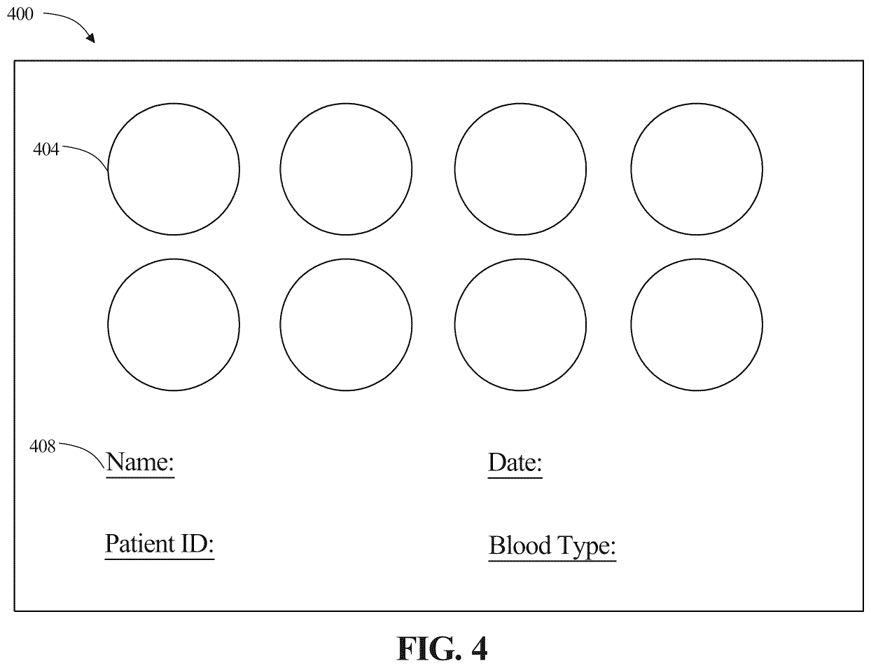

Referring now to FIG. 3D, yet another exemplary embodiment 300d of collection container 140 is described. In one or more embodiments collection container 140 may include and/or be fluidly connected to a test card 320. A “Test card” also known as a “dry blood spot card” for the purposes of this disclosure is a device that allows for collection and analysis of blood sample 116. In one or more embodiments, test card 320 may include a filter paper that is configured to store and dry blood samples 116 for further analysis. In one or more embodiments, test card 320 may include a material such as filter paper, blotting paper, and the like. In one or more embodiments, filter paper may include a porous paper that is used to separate solids from liquids. In one or more embodiments, the porosity of filter paper may be increased in comparison to ordinary printing paper wherein filter paper may allow for blood sample 116 to dry quicker. Test card 320 is described in further detail below such as in reference to at least FIG. 4. In one or more embodiments, collection container 140 may include test card. In one or more embodiments, collection container 140 may be fluidly connected to test card wherein a fluid from collection container 140 may be transmitted to test card through channels 324. In one or more embodiments, test card 320 may be configured to receive one or more droplets of blood sample 116 through the use of channels 324. In one or more embodiments, collection container may contain two compartments, a first compartment in which test card is stored and a second compartment in which blood sample 116 is received. In one or more embodiments, first compartment may be fluidly connected to second compartment wherein first compartment may contain openings and/or channels that allow for blood sample 116 to travel from first compartment to second compartment. In one or more embodiments, test card may receive one or more droplets of blood sample 116 in one or more predesigned areas of test card. In one or more embodiments, collection container 140 further includes test card, wherein test card is configured to receive one or more blood drops of blood sample 116. In one or more embodiments, collection container 140 may contain channels 324 configured to transport blood sample 116 to one or more portions of test card. In one or more embodiments, each channel 324 within collection container 140 may be configured to transport droplets to a particular portion of test card. In one or more embodiments, test card may include a plurality of predesigned areas wherein each predesignated area includes a portion of test card configured to a separate portion of blood sample 116 for review and analysis. For example, and without limitation, a first predesignated area may use blood drops of blood sample 116 to check for vitamin D levels, whereas a second predesignated area may be used to check for HDL levels. In one or more embodiments, collection container 140 may allow for the automated flow of blood sample 116 onto test card in order to prevent user contamination during placement of blood sample 116 onto test card. In one or more embodiments, an automated flow may include collection of blood sample into collection container 140 wherein blood sample may travel through channels 324 located within collection container and onto test card 320. In one or more embodiments, collection container 140 may serve as an enclosed casing for test card wherein test card may be isolated from debris and other foreign objects that can cause contamination. In one or more embodiments, placement of test card within collection container 140 may allow for faster placement of blood sample 116 onto test card. In one or more embodiments, blood sample 116 may first be collected by collection container 140 wherein channels 324 within collection container 140 may transport droplets of blood sample 116 to various portions of test card.

Referring now to FIG. 4, an exemplary embodiment of a test card 400 is described. In one or more embodiments, test card 400 may include a flat sheet or paper configured to receive one or more droplets fo blood sample 116. In one or more embodiments, test card 400 may include one or more predesignated areas 404, wherein each predesignated area 404 may be configured to receive a portion of blood sample 116. In one or more embodiments, collection container 140 may be used to transfer blood sample 116 onto test card 400. In one or more embodiments, collection container 140 may be in the form of a Pasteur Pipette may be used to place droplets onto test card 400. In one or more embodiments, collection container 140 may be in the form of syringe wherein user may control the flow of blood sample 116 onto test card 400. In one or more embodiments, collection container 140 may contain removable cap wherein removable cap may be removed for placement of blood sample 116 onto test card 400. In one or more embodiments, collection container 140 may contain test card 400 and/or a portion of test card 400 wherein blood drops of blood sample 116 may travel through channels and onto test card 400. In one or more embodiments, blood sample 116 may dry on test card 400 wherein blood sample 116 may be rehydrated for testing in laboratory. In one or more embodiments, test card 400 may include a material such as filter paper wherein the filter paper is configured to dry blood sample 116 for testing in laboratory. In one or more embodiments, drying of blood sample 116 onto test card 400 may allow for preservation of blood sample 116 prior to testing. In one or more embodiments test card 400 may be used to preserve blood sample 116. In one or more embodiments, blood sample 116 may be rehydrated in a laboratory wherein blood sample 116 may be tested for Enzymatic reactions, anti-body antigen reactions, DNA/RNA extraction, chemical assays and the like. In one or more embodiments, test card 400 may be inert and simply serve as a transfer medium for blood sample 116. In one or more embodiments, liquid blood may not be properly preserved and as a result, cannot be checked for various analytes. In one or more embodiments, dehydration of blood sample 116 on test card 400. In one or more embodiments, test card 400 may be transmitted to a laboratory for testing. In one or more embodiments, test card 400 may include identifying information 408 associated with blood sample 116, such as but not limited to, the user's name, date of collection, a patient ID if needed, a blood type and the like. In one or more embodiments, blood collection device 104 may allow for collection of blood sample 116 wherein blood sample 116 may be transferred to test card 400. In one or more embodiments, collection containers 140 may be removably attached to blood collection device 104, wherein a first collection container 140 may be used to receive blood sample 116 and transfer blood sample 116 onto test card 400 while a second collection container 140 may be used to preserve blood sample 116, wherein the preserved blood sample 116 may be transmitted to a laboratory for testing.

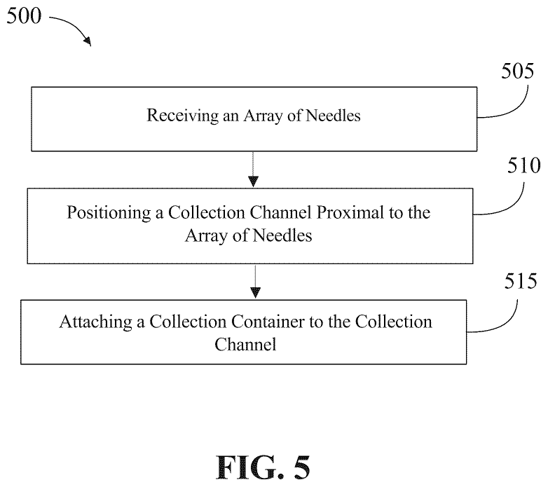

Referring now to FIG. 5, a method of manufacture 500 of a blood collection device is described. At step 505, method 500 includes receiving an array of needles configured to pierce an epidermal layer and extract a blood sample of a user. This may be implemented with reference to FIGS. 1-4 and without limitation.

With continued reference to FIG. 5, at step 510 method 500 includes positioning a collection channel proximal to the array of needles, wherein the collection channel is configured to transport the blood sample from the array of needles. This may be implemented with reference to FIGS. 1-4 and without limitation.

With continued reference to FIG. 5, at step 515 method 500 includes attaching a collection container to the collection channel wherein the collection container is configured to store the blood sample and wherein the collection container includes an inlet, wherein the inlet is configured to receive the blood sample and an outlet wherein the outlet is configured to expel the blood sample from the collection container. In one or more embodiments, the collection container includes a pliable material. In one or more embodiments the collection container includes a Pasteur pipette. In one or more embodiments the collection container further includes a removable cap located at the outlet. In one or more embodiments the removable cap is configured to prevent release of the blood sample from the outlet. In one or more embodiments the collection container is removably attached to the blood collection device. In one or more embodiments the collection container includes a syringe and wherein the syringe is configured to receive the blood sample at the inlet and expel the blood sample at the outlet. In one or more embodiments the method further includes mechanically connecting a push lever to the array of needles, wherein, when pressed, the push lever is configured to pierce the epidermal layer of the user using the array of needles. In one or more embodiments collection container further includes a test card configured to receive one or more blood drops of the blood sample. In one or more embodiments the collection container further includes one or more channels configured to transport the blood sample to one or more portions on the test card. This may be implemented with reference to FIGS. 1-4 and without limitation.

1. A blood collection device comprising:
an array of needles configured to pierce an epidermal layer and extract a blood sample of a user;
a collection channel positioned proximal to the array of needles and configured to transport the blood sample from the array of needles, wherein the collection channel is configured to enable blood flow by through a rotational mechanical work source; and
a collection container configured to store the blood sample, wherein the collection container comprises:
an inlet, wherein the inlet is configured to receive the blood sample; and
an outlet wherein the outlet is configured to expel the blood sample from the collection container.

2. The blood collection device of claim 1, wherein the collection container comprises a pliable material.

3. The blood collection device of claim 2, wherein the collection container comprises a Pasteur pipette.

4. The blood collection device of claim 1, wherein the collection container further comprises a removable cap located at the outlet.

5. The blood collection device of claim 4, wherein the removable cap is configured to prevent release of the blood sample from the outlet.

6. The blood collection device of claim 1, wherein the collection container is removably attached to the blood collection device.

7. The blood collection device of claim 1, wherein the collection container comprises a syringe and wherein the syringe is configured to receive the blood sample at the inlet and expel the blood sample at the outlet.

8. The blood collection device of claim 1, the blood collection device further comprising:
a push lever mechanically connected to the array of needles, wherein, when pressed, the push lever is configured to pierce the epidermal layer of the user using the array of needles.

9. The blood collection device of claim 1, wherein the collection container further comprises a test card configured to receive one or more blood drops of the blood sample.

10. The blood collection device of claim 9, wherein the collection container further comprises one or more channels configured to transport the blood sample to one or more portions on the test card.

11. A method of manufacture of a blood collection device comprising:
receiving an array of needles configured to pierce an epidermal layer and extract a blood sample of a user;
positioning a collection channel proximal to the array of needles, wherein the collection channel is configured to transport the blood sample from the array of needles, wherein the collection channel is configured to enable blood flow by through a rotational mechanical work source; and
attaching a collection container to the collection channel wherein the collection container is configured to store the blood sample and wherein the collection container comprises:
an inlet, wherein the inlet is configured to receive the blood sample; and
an outlet wherein the outlet is configured to expel the blood sample from the collection container.

12. The method of claim 11, wherein the collection container comprises a pliable material.

13. The method of claim 12, wherein the collection container comprises a Pasteur pipette.

14. The method of claim 11, wherein the collection container further comprises a removable cap located at the outlet.

15. The method of claim 14, wherein the removable cap is configured to prevent release of the blood sample from the outlet.

16. The method of claim 11, wherein the collection container is removably attached to the blood collection device.

17. The method of claim 11, wherein the collection container comprises a syringe and wherein the syringe is configured to receive the blood sample at the inlet and expel the blood sample at the outlet.

18. The method of claim 11, the method further comprising:
mechanically connecting, a push lever to the array of needles, wherein, when pressed, the push lever is configured to pierce the epidermal layer of the user using the array of needles.

19. The method of claim 11, wherein the collection container further comprises a test card configured to receive one or more blood drops of the blood sample.

20. The method of claim 19, wherein the collection container further comprises one or more channels configured to transport the blood sample to one or more portions on the test card.
